(19) United States
(12) Patent Application Publication
(10) Pub. No.: US 2022/0042985 A1
(43) Pub. Date: Feb. 10, 2022

(54) SARS-CoV-2 IgG/IgM ANTI-BODY DETECTION KIT

(71) Applicant: Tianjin Beroni Biotechnology Co., Ltd, Tianjin City (CN)

(72) Inventors: Boqing Zhang, Tianjin City (CN); Zhenghu Jia, Tianjin City (CN)

(21) Appl. No.: 16/987,049

(22) Filed: Aug. 6, 2020

(51) Int. Cl. G01N 33/558; G01N 33/569; B01L 3/00; G01N 33/538

(52) U.S. Cl. CPC G01N 33/558; G01N 33/56983; B01L 3/5027; B01L 2300/0825; G01N 2333/165; G01N 2469/20; G01N 33/538

(57) ABSTRACT

Disclosed is a SARS-CoV-2 IgG/IgM detection kit. The kit includes a disposable test cassette and the disposable test cassette includes a base plate, a sample pad, a reaction pad and an absorbent pad. The sample pad, the reaction pad and the absorbent pad are sequentially connected and are provided on the base plate. The sample pad is coated with a colloidal gold-labeled recombinant SARS-CoV-2 S-RBD protein. The reaction pad is provided with a test line and a quality control line. The test line is coated with a mouse anti-human IgG and a mouse anti-human IgM and the quality control line is coated with a goat anti-mouse IgG. The kit provided herein can be used for rapid test of SARS-CoV-2 IgG/IgM, and has high accuracy, good stability and strong anti-interference performance.

### TECHNICAL FIELD

This application relates to detection of antibodies, and more particularly to a SARS-CoV-2 IgG/IgM detection kit.

### BACKGROUND

CoronaVirus Disease 2019 (COVID-19) has an average incubation period of about 10 days, with a range from 1 day to about 14 days. The SARS-CoV-2 infected person still shows infectivity to others during the incubation period. Therefore, it is of significance to conduct effective screening as soon as possible for the containment of source of infection.

Currently, tests for SARS-CoV-2 is performed by collecting throat swabs or nasal swabs, but there is a risk of exposure since it is required to remove the mask during the collection. Furthermore, the nucleic acid testing by PCR usually uses PCR instrument, electrophoresis apparatus, etc., and the involved detection process is time-consuming, so it is not suitable for the large-scale early screening.

Given the above, there is an urgent need for persons skilled in the art to develop a kit allowing for rapid, accurate and stable detection and simple operation.

### SUMMARY

An object of this application is to provide a SARS-CoV-2 IgG/IgM detection kit to overcome the defects in the prior art.

The technical solutions of this application are described as follows.

This application provides a SARS-CoV-2 IgG/IgM detection kit, comprising:

a disposable test cassette;

wherein the disposable test cassette comprises a base plate, a sample pad, a reaction pad and an absorbent pad; the sample pad, the reaction pad and the absorbent pad are sequentially connected and are provided on the base plate;

the reaction pad is coated a colloidal gold-labeled recombinant SARS-CoV-2 S-RBD protein;

the reaction pad is provided with a test line and a quality control line; the test line is coated with a mouse anti-human IgG and a mouse anti-human IgM; and the quality control line is coated with a goat anti-mouse IgG.

In an embodiment, the test line is provided at a side of the reaction pad close to the sample pad; and the quality control line is provided at a side of the reaction pad close to the absorbent pad.

In an embodiment, the sample pad and the absorbent pad both are an absorbent paper; and the reaction pad is glass fiber membrane.

In an embodiment, the detection kit further comprises a dropper, a desiccant and a sample diluent.

In an embodiment, the sample diluent is a 0.01-0.015 M PBS with pH of 7.4±0.2.

After a human blood sample is added onto the sample pad, if there is SARS-CoV-2 IgG or IgM in the blood sample, the IgG or IgM antibody will bind to the colloidal gold-labeled antigen to form IgG or IgM colloidal gold immune complex, completing the labeling of the antibody in the sample. After the sample diluent is dropwise added, the immune complex is chromatographed upward under the action of the sample diluent. When the blood sample arrives at the test line, the immune complex of the colloidal gold-labeled antigen and IgG or IgM antibody will be captured by mouse anti-human IgG or IgM antibody to generate a red test line, indicating the presence of IgG or IgM antibody in the blood sample. Then the immune complex continues to move upward to arrive at the quality control line and binds to the goat anti-mouse IgG, generating a red quality control line and indicating that this detection is valid.

This application also provides a method of preparing the disposable test cassette of the above detection kit, comprising:

(1) spraying the colloidal gold-labeled recombinant SARS-CoV-2 S-RBD protein onto the sample pad followed by drying for use;

(2) coating the mouse anti-human IgG and the mouse anti-human IgM on the test line on the reaction pad; and coating the goat anti-mouse IgG on the quality control line;

(3) connecting the sample pad, the reaction pad and the absorbent pad sequentially followed by fixing on the base plate; cutting the base plate into strips; and encapsulating each strip into a shell to produce the disposable test cassette.

In an embodiment, the colloidal gold-labeled recombinant SARS-CoV-2 S-RBD protein is prepared through steps of:

(a) preparing a colloidal gold solution;

(b) diluting a recombinant SARS-CoV-2 S-RBD protein with a buffered solution; adjusting the colloidal gold solution to pH 9.0-9.2; adding the diluted S-RBD protein solution to the colloidal gold solution followed by standing; adding a bovine serum albumin (BSA) solution to the reaction mixture followed by stirring; and centrifuging the reaction mixture;

(c) after the centrifugation is completed, collecting a first precipitate; dissolving the first precipitate with a first tris-(hydroxymethyl)-aminomethane (Tris) solution containing BSA followed by centrifugation to collect a second precipitate; and dissolving the second precipitate with a second Tris solution comprising sodium azide, sucrose and BSA to produce the colloidal gold-labeled recombinant SARS-CoV-2 S-RBD protein.

In an embodiment, in step (a), colloidal gold in the colloidal gold solution has a particle size of 20-40 nm.

In an embodiment, in step (b), the buffered solution is a 0.1-0.15 mM PBS; the recombinant SARS-CoV-2 S-RBD protein in the mixture of the diluted S-RBD protein solution and the colloidal gold solution has a concentration of 10-15 μg/mL; and BSA in the BSA solution has a mass percentage of 10-15%.

In an embodiment, in step (c), the first Tris solution has a BSA mass percentage of 1-1.5%, a Tris concentration of 0.01-0.02 mol/L and pH of 8.0-8.3;

the second Tris solution has a BSA mass percentage of 1-1.5%, a sodium azide mass percentage of 0.01-0.02%, a sucrose mass percentage of 0.8-1.2%, a Tris concentration of 0.01-0.02 mol/L and pH of 8.0-8.3.

Compared to the prior art, the invention has the following beneficial effects.

The detection kit provided herein for in vitro rapid diagnosis of SARS-CoV-2 is prepared based on colloidal gold immunochromatography, in which anti-human IgG and IgM antibodies are used as coating materials, and the artificially-purified SARS-CoV-2 specific antigen is used as a labeling material. The kit has simple operation, high accuracy, good specificity, strong detection and storage stability and excellent anti-interference, and thus it is suitable for the detection of whole blood, serum and plasma samples, contributing to the large-scale screening of SARS-CoV-2.

### BRIEF DESCRIPTION OF THE DRAWINGS

In order to make the technical solutions in this application or the prior art clearer, the accompanying drawings used in the following embodiments will be briefly described below. Obviously, the following accompanying drawings merely illustrate some embodiments of the invention, and those skilled in the art can obtain other drawings based on these drawings without sparing any creative effort.

FIG. 1 schematically illustrates a disposable test cassette of a SARS-CoV-2 IgG/IgM detection kit according to the invention, where T: test line; C: quality control line; and S-sample hole.

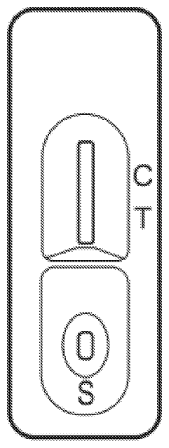

### DETAILED DESCRIPTION OF EMBODIMENTS

The technical solutions of the invention will be clearly and completely described below with reference to the accompanying drawings and embodiments. Obviously, described below are merely some embodiments of the invention, which are not intended to limit the invention. Other embodiments obtained by those skilled in the art based on the following embodiments without sparing any creative effort shall fall within the scope of the invention.

Retrospective clinical specimens collected from COVID-2019 (Coronavirus disease) patients confirmed by RT-PCR are used in the following embodiments to conduct the clinical research on the SARS-CoV-2 test. The recombinant SARS-CoV-2 S-RBD protein (YP_009724390.1, Arg319-Phe541, mFc Tag), mouse anti-human IgG [Human IgG-MAb (SARS-CoV-2)], mouse anti-human IgM [Anti-Human IgM monoclonal (SARS-CoV-2)] and goat anti-mouse IgG and other materials are purchased from Shanghai Jieyi Biotechnology Co., Ltd.

### Example 1

Provided herein was a SARS-CoV-2 IgG/IgM detection kit, including a disposable test cassette, a dropper, a desiccant and a sample diluent.

The disposable test cassette included a base plate, a sample pad, a reaction pad and an absorbent pad, where the sample pad, the reaction pad and the absorbent pad were sequentially connected and were provided on the base plate. The reaction pad was coated a colloidal gold-labeled recombinant SARS-CoV-2 S-RBD protein. The reaction pad was provided with a test line and a quality control line, where the test line was coated with a mouse anti-human IgG and a mouse anti-human IgM, and the quality control line was coated with a goat anti-mouse IgG. The test line was provided at a side of the reaction pad close to the sample pad, and the quality control line was provided at a side of the reaction pad close to the absorbent pad. The sample pad and the absorbent pad both were an absorbent paper, and the reaction pad was a glass fiber membrane.

The disposable test cassette, the dropper and the desiccant were packaged in a foil pouch.

The sample diluent was a 0.01-0.015 M PBS with pH of 7.4±0.2.

The disposable test cassette was prepared as follows.

(1) Preparation of the colloidal gold-labeled recombinant SARS-CoV-2 S-RBD protein

(1.1) Preparation of a colloidal gold solution

A 0.2% aqueous chloroauric acid solution was heated to 95° C., quickly added with a 1.5% aqueous trisodium citrate solution and continuously stirred until the reaction mixture became burgundy to produce the colloidal gold solution in which the colloidal gold had a particle size of 20-40 nm, which was stored at 4° C. for use.

(1.2) The recombinant SARS-CoV-2 S-RBD protein was diluted to 0.5 mg/mL with 0.12 mM phosphate buffer (PB). The colloidal gold solution was adjusted to pH 9.0, and the diluted protein solution was added to 10 mL of the colloidal gold solution to a final recombinant SARS-CoV-2 S-RBD protein concentration of 20 μg/mL. The reaction mixture was subjected to standing for 5 min, added with a 10% BSA solution, stirred and centrifuged.

(1.3) After the centrifugation was completed, a precipitate was collected and dissolved with a first Tris solution containing BSA. The resulting solution was centrifuged, and the precipitate was collected and dissolved with second Tris solution comprsising sodium azide, sucrose and BSA to produce the colloidal gold-labeled recombinant SARS-CoV-2 S-RBD protein.

The first Tris solution had a BSA mass percentage of 1%, a Tris concentration of 0.015 mol/L and pH of 8.0.

The second Tris solution had a BSA mass percentage of 1.2%, a sodium azide mass percentage of 0.01%, a sucrose mass percentage of 1%, a Tris concentration of 0.015 mol/L and pH of 8.0.

(2) The colloidal gold-labeled recombinant SARS-CoV-2 S-RBD protein was sprayed on the sample pad with a spraying membrane instrument and dried at 37° C. for 2 h.

(3) 0.8 mg/mL mouse anti-human IgG and 0.8 mg/mL mouse anti-human IgM were sprayed on the test line with the spraying membrane instrument, and 0.8 mg/mL was sprayed on the quality control line at an amount of 20 μL/10 cm. Then the test line and the quality control line were dried at 37° C. for 2 h.

(4) The sample pad, the reaction pad and the absorbent pad were sequentially connected and fixed on the base plate, and the base plate was cut into multiple strips each with a width of 3 mm. Each strip was encapsulated in a shell to produce the disposable test cassette with a liquid migration rate equal to or more than 10 mm/min, where the shell was provided with a sample hole S and an observation window, and the color changes at the test line T and the quality control line C can be observed through the observation window.

SARS-CoV-2 IgG/IgM detection kit was used as follows.

The kit was equilibrated to room temperature before use. One drop of a blood sample (approximately 30 μL) was added to the sample hole, and 5 seconds later, three drops of the sample diluent (approximately 120 μL) were added to the sample hole. Then the test cassette was placed on a flat surface, and the results were observed through the observation window after 10 min.

In the case that the quality control line C and the test line T were both observable, it indicated positive results, that was, IgG/IgM were present in the sample.

In the case that only the quality control line C was observable, it indicated negative results, that was, the SARS-CoV-2 IgG and IgM were not detected in the sample.

In the case that the quality control line C was not observable, it indicated that this detection was invalid regardless of whether there were other observable lines in the test cassette, and the detection should be repeated using a new test cassette.

### Example 2 Investigation on Detection Limit

A serum sample G collected from a mild patient tested positive for SARS-CoV-2 was employed to estimate the detection limit of the SARS-CoV-2 IgG/IgM detection kit prepared in Example 1.

The serum sample G was diluted in three different dilutions (respectively 1:250, 1:100 and 1:25) to accordingly produce three diluted samples G1, G2 and G3. Each of the diluted samples G1, G2 and G3 was detected in triplicate respectively using three independent batches of the SARS-CoV-2 IgG/IgM detection kit (8420200204, 8420200205 and 8420200206).

The detection results were shown in the FIGURE, and it can be observed that the SARS-CoV-2 IgG/IgM were detected both in the samples G2 and G3 (9/9), and not detected in the sample G1 (0/9). Therefore, it can be estimated that the detection limit for the SARS-CoV-2 positive serum sample from a mild patient was the level of IgG/IgM in the sample diluted in 1:100.

TABLE 1
Detection results for the SARS-CoV-2 positive
serum samples with different dilutions

| | Results | | |
|---|---|---|---|
| | Batch number | | |
| Samples | 8420200204 | 8420200205 | 8420200206 |
| G1 | 0/3 | 0/3 | 0/3 |
| G2 | 3/3 | 3/3 | 3/3 |
| G3 | 3/3 | 3/3 | 3/3 |

### Example 3 Repeatability Testing

The SARS-CoV-2 IgG/IgM detection kit prepared in Example 1 was subjected to repeatability test.

1. Single positive and negative reference samples were used to evaluate the repeatability of the test, and each sample was tested 10 times.

TABLE 2
Repeatability test

| | Batch | | | | | |
|---|---|---|---|---|---|---|
| | 8420200204 | | 8420200205 | | 8420200206 | |
| | Positive | Negative | Positive | Negative | Positive | Negative |
| No. | reference | reference | reference | reference | reference | reference |
| 1 | + | − | + | − | + | − |
| 2 | + | − | + | − | + | − |
| 3 | + | − | + | − | + | − |
| 4 | + | − | + | − | + | − |
| 5 | + | − | + | − | + | − |
| 6 | + | − | + | − | + | − |
| 7 | + | − | + | − | + | − |
| 8 | + | − | + | − | + | − |
| 9 | + | − | + | − | + | − |
| 10 | + | − | + | − | + | − |

It can be seen from Table 2 that the detection repeatability for the SARS-CoV-2 IgG/IgM positive and negative serum samples using the kit provided herein was 100%.

2. 25 serum samples, 25 plasma samples and 25 whole blood samples from volunteers tested negative for SARS-CoV-2 and 25 serum samples, 25 plasma samples and 25 whole blood samples from patients tested positive for SARS-CoV-2 were collected, and each of the above samples was detected three times for the SARS-CoV-2 IgG/IgM respectively using different batches of the detection kits (8420200204, 8420200205 and 8420200206) to evaluate the repeatability of the detection for IgG/IgM in the serum samples, plasma samples and whole blood samples.

The results were shown in Table 3, and it can be obtained that the detection for IgG/IgM whether in the SARS-CoV-2 negative or positive samples showed a consistency of 100%.

TABLE 3
Repeatability of the detection of IgG/IgM in serum, plasma and whole blood

| | | | Results | | | |
|---|---|---|---|---|---|---|
| SARS-CoV-2 | Number of | Batch number | | | Whole | |
| negative or | samples | of the kit | Serum | Plasma | blood | Consistency |
| Negative | 25 | 8420200204 | 0/25 | 0/25 | 0/25 | 100% |
| | 25 | 8420200205 | 0/25 | 0/25 | 0/25 | 100% |
| | 25 | 8420200206 | 0/25 | 0/25 | 0/25 | 100% |
| Positive | 25 | 8420200204 | 25/25 | 25/25 | 25/25 | 100% |
| | 25 | 8420200205 | 25/25 | 25/25 | 25/25 | 100% |
| | 25 | 8420200206 | 25/25 | 25/25 | 25/25 | 100% |

Further, serum, plasma and whole blood samples of one SARS-CoV-2 positive patient were collected and subjected to 8, 12, 16, 20, 24, 28, 32, 40, 44, 48, 52, 56, 60, 64, 68, 72, 76, 80, 84, 88, 92, 96 and 100-fold dilutions with PBS (0.01M, pH 7.4±0.2), respectively. The diluted serum, plasma and whole blood samples were all tested positive for SARS-CoV-2.

Serum, plasma and whole blood samples of one SARS-CoV-2 negative volunteer were correspondingly added with 1% positive sample and then detected for the IgG/IgM. The results showed that all samples were tested negative before addition, and tested positive after the addition.

3. High-dose hook effect

Whether the hook effect will occur in the detection of SARS-CoV-2 IgG or IgM using the SARS-CoV-2 IgG/IgM detection kit prepared herein was evaluated by increasing the purified IgG/IgM dose in the positive reference sample (10000×, 20000× and 30000×).

The test results were shown in Table 4, and it can be seen that there was no decline in the intensity of the detection for SARS-CoV-2 IgG or IgM, which indicated that the SARS-CoV-2 IgG/IgM detection kit of the invention will not generate the high-dose hook effect or prozone effect.

TABLE 4
Test for the high-dose hook effect

| | | SARS-CoV-2 IgG/IgM detection kit batch number | | | | | | | | |
|---|---|---|---|---|---|---|---|---|---|---|
| | | 8420200204 | | | 8420200205 | | | 8420200206 | | |
| Positive | | Repeated test | | | | | | | | |
| reference sample | Concentration | 1 | 2 | 3 | 1 | 2 | 3 | 1 | 2 | 3 |
| High-concentration | 10000× | + | + | + | + | + | + | + | + | + |
| SARS-CoV-2 IgG | 20000× | + | + | + | + | + | + | + | + | + |
| | 30000× | + | + | + | + | + | + | + | + | + |
| High-concentration | 10000× | + | + | + | + | + | + | + | + | + |
| SARS-CoV-2 IgM | 20000× | + | + | + | + | + | + | + | + | + |
| | 30000× | + | + | + | + | + | + | + | + | + |

### Example 4 Investigation of Cross Reaction

Whether a cross reaction caused by other pathogens and components that may lead to the occurrence of a cross reaction will occur during the use of the detection kit of the invention was evaluated as follows.

1. Three batches of the detection kits prepared in Example 1 (8420200204, 8420200205 and 8420200206) were used to detect the negative serum samples respectively added with other pathogens (each for 3 concentrations: 50 pfu/mL, 100 pfu/mL and 200 pfu/mL) or pathogen antibodies (each for 3 concentrations: 25 mIU/L, 50 mIU/L and 100 mIU/L), respectively. The pathogen was mycoplasma pneumonia, and the pathogen antibodies were listed as follows:

(1) endemic human coronavirus HKU1, OC43, NL63 and 229E;

(2) H1N1 ((the new H1N1 (2009) and seasonal H1N1), H3N2, H5N1, H7N9, influenza B Yamagata, Victoria, respiratory syncytial virus, rhinovirus A, B and C, adenovirus 1, 2, 3, 4, 5, 7 and 55, enterovirus A, B, C and D, EB virus, measles virus, human cytomegalovirus, rotavirus, norovirus, mumps virus and varicella-zoster virus.

Each pathogen or pathogen antibody was added separately and tested as a sample.

The results revealed that the serum samples whether added with the pathogen (50 pfu/mL, 100 pfu/mL and 200 pfu/mL) or the above pathogen antibodies (25 mIU/L, 50 mIU/L and 100 mIU/L) were tested negative for SARS-CoV-2 IgG/IgM, which indicated that there was no cross reaction with the kit of the invention.

2. Three batches of the detection kits prepared in Example 1 (8420200204, 8420200205 and 8420200206) were used to detect the samples added with the following interfering substances: the samples included negative samples and weak positive samples and the samples from the same source set as serum samples, plasma samples and whole blood samples respectively. In other words, each concentration of interfering substances was respectively added to six sample groups (negative serum group, negative plasma group, negative whole blood group, weak positive serum group, weak positive plasma group and weak positive whole blood group) for detection. A control group was set without the addition of interfering substances.

The interfering substances included purified mucin (10 mg/mL and 20 mg/mL), bilirubin (10 mg/mL and 20 mg/mL), triglyceride (150 ng/mL and 200 ng/mL), hemoglobin (10 mg/mL and 20 mg/mL), rheumatoid factor (80 IU/mL and 160 IU/mL), antinuclear antibody (100 IU/mL and 200 IU/mL), anti-mitochondrial antibody (100 IU/mL and 200 IU/mL), heterophilic antibody (HAMA, 100 IU/mL and 200 IU/mL), total IgG (15 g/L and 30 g/L), total IgM (2 g/L and 4 g/L), hematocrit (for whole blood samples, 0.35 and 0.5), α-interferon (1.5 mg/L and 3 mg/L), zanamivir (0.1 μg/mL and 0.2 μg/mL), ribavirin (10 μg/mL and 20 μg/mL), oseltamivir (1.5 μg/mL and 3.0 μg/mL), paramivir (10 μg/mL and 20 μg/mL), lopinavir (8 μg/mL and 16 μg/mL), ritonavir (2 μg/mL and 4 μg/mL), arbidol (4 μg/mL and 8 μg/mL), levofloxacin (4 μg/mL and 8 μg/mL), azithromycin (10 μg/mL and 20 μg/mL), ceftriaxone (40 μg/mL and 80 μg/mL), meropenem (10 μg/mL and 20 μg/mL), tobramycin (1.5 μg/mL and 3.0 μg/mL), histamine hydrochloride (10 pg/mL and 20 pg/mL), phenylephrine (0.1 μg/mL and 0.2 μg/mL), oxymetazoline (0.25 g/L and 0.5 g/L), sodium chloride (containing a preservative, 0.8% and 0.9%), beclomethasone (0.02 g/mL and 0.04 g/mL), dexamethasone (0.05 g/mL and 0.10 g/mL), flunisolide (0.01 g/mL and 0.02 g/mL), triamcinolone acetonide (1.5 μg/mL and 3.0 μg/mL), budesonide (8 ng/mL and 16 ng/mL), mometasone (4 ng/mL and 8 ng/mL) and fluticasone (2 ng/mL and 4 ng/mL).

The results demonstrated that all weak positive samples were tested positive and all negative samples were tested negative.

### Example 5 Investigation of Stability

1. High-Temperature Acceleration Stability

Three batches of the detection kits prepared in Example 1 (8420200204, 8420200205 and 8420200206) were placed at 45° C. for 7, 14, 21, 28, 35 and 42 days, respectively, and then were tested for the physical properties, detection limit and repeatability.

The results revealed that after placed for 7-42 days, these kits all showed a liquid migration rate of 19-21 mm/min; the negative samples were still tested negative; the samples G2 and G3 in Example 2 were still tested positive, and the sample G1 was still tested negative; and 10 repeated tests for the positive sample all displayed positive results. Therefore, the kit provided herein met the related technical requirements after placed at 45° C. for 42 days.

The detection performance of the kit after stored at 2-30° C. for 1 year was equivalent to that of the kit aged at 45° C. for 37 days.

2. Transport Stability

Three batches of the detection kits prepared in Example 1 (8420200204, 8420200205 and 8420200206) were placed in a shaker and shaken for 8, 16, 24 and 32 days, respectively, and then were tested for the physical properties, detection limit and repeatability.

It can be seen from the results that after shaken for 8-32 days, these kits all showed a liquid migration rate of 19-21 mm/min; the negative samples were still tested negative; the samples G2 and G3 in Example 2 were still tested positive, and the sample G1 was still tested negative; and 10 repeated tests for the positive sample all displayed positive results. Given the above, the kit provided herein still enabled stable detection after shaken for 32 days, which indicated that it can still provide reliable detection results even considering the international transportation and some extreme conditions that may be encountered.

3. Stability after Opened

Three batches of the detection kits prepared in Example 1 (8420200204, 8420200205 and 8420200206) were opened and placed under different humidity conditions (≤30%, 30-65% and ≥65%) for 1 and 2 h, respectively, and then were tested for the physical properties, detection limit and repeatability.

The results revealed that after placed under different humidity conditions for 1 or 2 h, these kits all showed a liquid migration rate of 19-21 mm/min; the negative samples were still tested negative; the samples G2 and G3 in Example 2 were still tested positive, and the sample G1 was still tested negative; and 10 repeated tests for the positive sample all displayed positive results.

Since the test cassette may suffer dampness after the kit kept open for more than 1 h, it should be used within 1 h after opened.

4. Sample Stability

Three batches of the detection kits prepared in Example 1 (8420200204, 8420200205 and 8420200206) were used to detect the samples which had undergone different treatments.

(1) Detection stability for samples stored at 2-8° C.

1) Whole blood

The whole blood samples were collected from a patient tested positive for SARS-CoV-2, placed at 2-8° C. and tested after 0 (tested immediately), 1, 2, 3 and 4 days, respectively. The six samples were all tested positive after stored at 2-8° C. for 0-3 days, and some samples were tested negative after stored for 4 days.

Therefore, the kit of the invention can provide reliable detection with respect to a whole blood sample stored at 2-8° C. for no more than 3 days.

2) Serum

The whole blood samples were collected from a patient tested positive for SARS-CoV-2 and centrifuged immediately to collect the serum samples, which were placed at 2-8° C. and tested after 0 (tested immediately), 1, 2, 3, 4, 5, 6, 7 and 8 days, respectively. The six serum samples were all tested positive after stored at 2-8° C. for 0-7 days, and some samples were tested negative after stored for 8 days.

Therefore, the kit of the invention can provide reliable detection with respect to a serum sample stored at 2-8° C. for no more than 7 days.

3) Plasma

The whole blood samples were collected from a patient tested positive for SARS-CoV-2 and centrifuged immediately to collect the plasma samples, which were placed at 2-8° C. and tested after 0 (tested immediately), 1, 2, 3, 4, 5, 6, 7 and 8 days, respectively. The six plasma samples were all tested positive after stored at 2-8° C. for 0-7 days, and some samples were tested negative after stored for 8 days.

Therefore, the kit of the invention can provide reliable detection with respect to a plasma sample stored at 2-8° C. for no more than 7 days.

(2) Detection stability for samples stored at −20° C.

1) Whole blood

Three whole blood samples from positive patients and three whole blood samples from volunteers tested negative were collected and tested immediately. Simultaneously, the whole blood samples were individually placed in a test tube, stored at −20° C. and tested 1 day later. On the day of the sampling, the kit exhibited accurate test results for all of the six samples, and after stored at −20° C. for 1 day, some positive samples were tested negative and the negative samples were still tested negative.

Given the above, the detection kit of the invention failed to maintain a reliable detection performance for the whole blood sample stored at −20° C.

2) Serum

Six whole blood samples were collected from positive patients were centrifuged immediately to collect the serum samples, which were tested immediately and simultaneously placed individually in a test tube, stored at −20° C. for 1 month and restored to room temperature for test. The kit of the invention showed accurate test results for the six samples whether on the sampling day or after stored for 1 month.

Therefore, the detection performance of the kit provided herein can be maintained for at least one month for the serum sample stored at −20° C.

3) Plasma

Six whole blood samples were collected from positive patients were centrifuged immediately to collect the plasma samples, which were tested immediately and simultaneously placed individually in a test tube, stored at −20° C. for 1 month and restored to room temperature for test. The kit of the invention showed accurate test results for the six samples whether on the sampling day or after stored for 1 month.

Therefore, the detection performance of the kit provided herein can be maintained for at least one month for the plasma sample stored at −20° C.

(3) Detection stability for repeatedly frozen-thawed samples

1) Whole blood

Three whole blood samples from positive patients and three whole blood samples from volunteers tested negative were collected and tested immediately. Simultaneously, the whole blood samples were individually placed in a test tube, frozen at −20° C., thawed and restored to room temperature for test. On the day of the sampling, the kit exhibited accurate test results for all of the six samples, and after frozen and thawed, the positive samples were tested negative and the negative samples were still tested negative.

Therefore, the kit provided herein failed to accurately detect the frozen-thawed whole blood sample.

2) Serum

Six whole blood samples were collected from positive patients were centrifuged immediately to collect the serum samples, which were tested immediately and simultaneously placed individually in a test tube, stored at −20° C., subjected to freezing-thawing 1, 2 and 3 times respectively, and restored to room temperature for test. In the case that no freezing-thawing treatment was performed or the freezing-thawing treatment was performed less than or equal to 2 times, the kit of the invention showed accurate test results for the six samples. After frozen and thawed three times, some positive samples were tested negative.

Therefore, the kit provided herein can provide reliable detection performance for the serum sample stored at −20° C. and experiencing freezing and thawing 2 times or less, and the repeated freezing and thawing should be avoided in the actual test.

3) Plasma

Six whole blood samples were collected from positive patients were centrifuged immediately to collect the plasma samples, which were tested immediately and simultaneously placed individually in a test tube, stored at −20° C., subjected to freezing-thawing 1, 2 and 3 times, respectively, and restored to room temperature for test. In the case that no freezing-thawing treatment was performed or the freezing-thawing treatment was performed less than or equal to 2 times, the kit of the invention showed accurate test results for the six samples. After frozen and thawed three times, some positive samples were tested negative.

Therefore, the kit provided herein can provide reliable detection performance for the plasma sample stored at −20° C. and experiencing freezing and thawing 2 times or less, and the repeated freezing and thawing should be avoided in the actual test.

In summary, the kit of the invention can be stored at 2-30° C. for 12 months; for the detection of SARS-CoV-2 antibodies, the whole blood sample can be stored at 2-8° C. for 3 days, and should not be frozen; the serum sample and the plasma sample can be stored at 2-8° C. for 7 days and at −20° C. for 1 month, and should not be repeatedly frozen and thawed.

It should be noted that the above mentioned embodiments can be freely combined as needed. These embodiments are intended to enable those skilled in the art to implement the invention, and are not intended to limit the invention. Various modifications can be made by those skilled in the art based on the content disclosed herein without departing from the spirit of the invention and the scope of the invention.

What is claimed is:

1. A SARS-CoV-2 IgG/IgM detection kit, comprising a disposable test cassette, comprising: a base plate, a sample pad, a reaction pad, and an absorbent pad;
wherein the sample pad, the reaction pad and the absorbent pad are sequentially connected and are provided on the base plate;
the sample pad is coated a colloidal gold-labeled recombinant SARS-CoV-2 S-RBD protein;
the reaction pad is provided with a test line and a quality control line; the test line is coated with a mouse anti-human IgG and a mouse anti-human IgM; and the quality control line is coated with a goat anti-mouse IgG.

2. The detection kit of claim 1, wherein the test line is provided at a side of the reaction pad close to the sample pad; and the quality control line is provided at a side of the reaction pad close to the absorbent pad.

3. The detection kit of claim 1, wherein the sample pad and the absorbent pad both are an absorbent paper; and the reaction pad is a glass fiber membrane.

4. The detection kit of claim 1, further comprising: a dropper, a desiccant and a sample diluent.

5. The detection kit of claim 4, wherein the sample diluent is a 0.01-0.015 M PBS with pH of 7.4±0.2.

6. A method of preparing the disposable test cassette of the detection kit of claim 1, comprising:
(1) spraying the colloidal gold-labeled recombinant SARS-CoV-2 S-RBD protein onto the sample pad followed by drying for use;
(2) coating the mouse anti-human IgG and the mouse anti-human IgM on the test line on the reaction pad; and coating the goat anti-mouse IgG on the quality control line;
(3) connecting the sample pad, the reaction pad and the absorbent pad sequentially followed by fixing on the base plate; cutting the base plate into strips; and encapsulating each strip into a shell to produce the disposable test cassette.

7. The method of claim 6, wherein the colloidal gold-labeled recombinant SARS-CoV-2 S-RBD protein is prepared through steps of:
(a) preparing a colloidal gold solution;
(b) diluting a recombinant SARS-CoV-2 S-RBD protein with a buffered solution; adjusting the colloidal gold solution to pH 9.0-9.2; adding the diluted S-RBD protein solution to the colloidal gold solution followed by standing; adding a bovine serum albumin (BSA) solution to the reaction mixture followed by stirring; and centrifuging the reaction mixture;
(c) after the centrifugation is completed, collecting a first precipitate; dissolving the first precipitate with a first tris-(hydroxymethyl)-aminomethane (Tris) solution containing BSA followed by centrifugation to collect a second precipitate; and dissolving the second precipitate with a second Tris solution comprising sodium azide, sucrose and BSA to produce the colloidal gold-labeled recombinant SARS-CoV-2 S-RBD protein.

8. The method of claim 7, wherein in step (a), colloidal gold in the colloidal gold solution has a particle size of 20-40 nm.

9. The method of claim 7, wherein the buffered solution is a 0.1-0.15 mM PBS; the recombinant SARS-CoV-2 S-RBD protein in the mixture of the diluted S-RBD protein solution and the colloidal gold solution has a concentration of 10-15 μg/mL; and BSA in the BSA solution has a mass percentage of 10-15%.

10. The method of claim 7, wherein the first Tris solution has a BSA mass percentage of 1-1.5%, a Tris concentration of 0.01-0.02 mol/L and pH of 8.0-8.3;
The second Tris solution has a BSA mass percentage of 1-1.5%, a sodium azide mass percentage of 0.01-0.02%, a sucrose mass percentage of 0.8-1.2%, a Tris concentration of 0.01-0.02 mol/L and pH of 8.0-8.3.
